(19) United States
(12) Patent Application Publication
(10) Pub. No.: US 2025/0235218 A1
(43) Pub. Date: Jul. 24, 2025

(54) MULTI-AXIS CONFIGURABLE CANNULATION GUIDE

(71) Applicant: Zimmer, Inc., Warsaw, IN (US)

(72) Inventors: Jacob Hiday, Ann Arbor, MI (US); Jaime D. Winchester, Warsaw, IN (US); Tyler Sederlund, Columbia City, IN (US); Jon Lepage, Warsaw, IN (US)

(21) Appl. No.: 19/022,891

(22) Filed: Jan. 15, 2025

Related U.S. Application Data
(60) Provisional application No. 63/622,789, filed on Jan. 19, 2024.

(51) Int. Cl. A61B 17/17; A61B 17/34; A61B 17/86

(52) U.S. Cl. CPC A61B 17/1778; A61B 17/3423; A61B 17/864

(57) ABSTRACT

A medical device to facilitate the formation of an aperture at a target site is provided. The medical device includes a base, a coupling operatively coupled to the base, and a cannulated pin guide. The base has a cannula and a flange extending from a surface of the base where the flange includes a slot. The coupling has a tab extending therefrom and into the base flange where the coupling defines a slot. The cannulated pin guide extends through the coupling slot and into the base cannula.

### CLAIM OF PRIORITY

This application claims the benefit of U.S. Provisional Patent Application Ser. No. 63/622,789, filed on Jan. 19, 2024, the benefit of priority of which is claimed hereby, and which is incorporated by reference herein in its entirety.

### TECHNICAL FIELD

This document pertains generally, but not by way of limitation, to a positioning guide that can be used during medical procedures. More specifically, but not by way of limitation, this document relates to a positioning guide for glenoid placement during reverse shoulder replacement.

### BACKGROUND

A patient may suffer from shoulder joint pain or have a limited range of shoulder motion when various conditions exist, such as arthritis, avascular necrosis, or humeral head fractures, to name a few. When conservative treatment including medications, physical therapy, injections, or the like, fail to remedy shoulder joint pain or increase range of motion, reverse shoulder joint replacement can be a viable option.

Reverse shoulder replacement can involve placing an implant at the glenoid of the scapula. Typically, in order to position the implant at the glenoid, a pin can be inserted into the glenoid at the glenoid cavity. The pin can be used to orient a glenosphere in the glenoid cavity. Moreover, the pin can be placed at an angle such that when the glenosphere is implanted using the pin, the glenosphere can extend from the glenoid cavity at the angle at which the pin was placed. A practitioner, such as a surgeon, free hands the angle without any way to maintain the angle during reaming of the scapula. However, the angle may change without the practitioner realizing the change. Thus, the angle is not maintained during reaming such that the implant can be improperly placed.

Furthermore, three-dimensional (3D) guides can be created which have an angle preferred by a practitioner. Moreover, a 3D guide can be customized for a patient receiving the 3D guide. Thus, if the practitioner prefers that a glenosphere extends from the glenoid cavity at an angle, the guide can be 3D printed to include the angle. Here, when the guide is placed within the glenoid cavity and the glenosphere is implanted, the glenosphere will extend from the glenoid cavity at the angle. However, the angle is static. Thus, a practitioner cannot change the angle at which the glenosphere extends from the glenoid cavity in-situ.

### SUMMARY

Accordingly, what is needed is a guide that can allow a practitioner to dial in an angle at which a glenosphere will extend from a bone in-situ. A cannulation guide is provided that includes a base configured to provide two degrees of freedom for a cannulated pin guide that can be used to allow for selection and locking of an angle at which a target site can be reamed. The cannulation guide can include a coupling having a tab operatively coupled with the base where the coupling can be configured to receive the cannulated pin guide. The cannulated pin guide can pass through the coupling and into the base. The base can be configured to allow for movement of the coupling in a first direction while at the same time allow for movement of the cannulated pin guide in a second direction different from the first direction, thereby providing two degrees of freedom.

The base can include a slot that receives the coupling tab such that the coupling tab can move within the base slot in the first direction. The coupling can include a slot that receives the cannulated pin guide where the cannulated pin guide can extend from the coupling slot and into the base. The base can include a cannula through which the cannulated pin guide can extend. The cannulated pin guide can have a first diameter and the base cannula can have a second diameter greater than the first diameter. Thus, the cannulated pin guide can move within the base cannula. By virtue of the base having the base slot that receives the coupling tab and the base having the base cannula that receives the cannulated pin guide, the cannulation guide can provide a practitioner, such as a surgeon, the ability to dial in an angle at which a glenosphere will extend from a bone in-situ.

### BRIEF DESCRIPTION OF FIGURES

FIG. 1 illustrates an environment within which examples can be used.

FIG. 2 is a schematic of a cannulation guide, in accordance with some examples.

FIGS. 3A and 3B illustrate a base of the cannulation guide of FIG. 2, in accordance with some examples.

FIG. 4 shows a coupling member of the cannulation guide of FIG. 2, in accordance with some examples.

FIG. 5 illustrates a base slot of the base of FIG. 3A, in accordance with some examples.

FIG. 6 shows another view of the coupling member of the cannulation guide of FIG. 2, in accordance with some examples.

FIG. 7 shows a set screw of the cannulation guide of FIG. 2, in accordance with some examples.

FIG. 8 illustrates another view of the base of FIGS. 3A and 3B, in accordance with some examples.

FIG. 9 illustrates a cannulated pin guide of the cannulation guide of FIG. 2, in accordance with some examples.

FIG. 10 illustrates a cannulated pin guide handle of the cannulation guide of FIG. 2, in accordance with some examples.

FIG. 11 illustrates a method for using a cannulation guide to form an aperture at a target site, in accordance with some examples.

### DETAILED DESCRIPTION

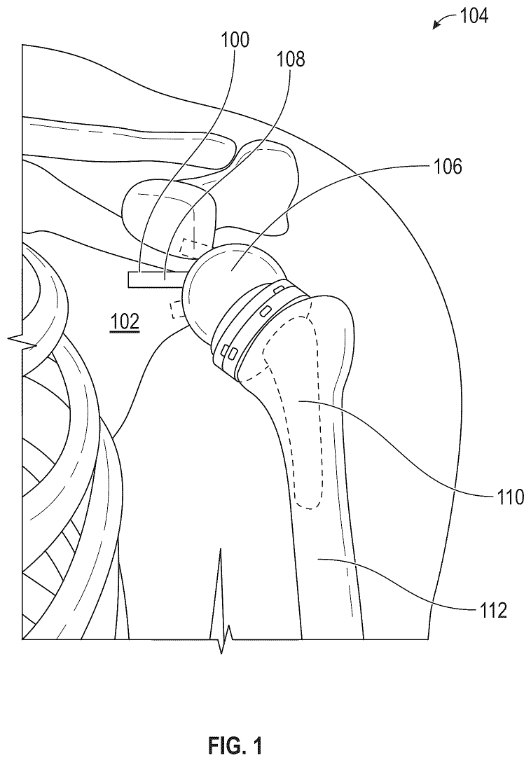

A cannulation guide is provided that includes a base configured to provide two degrees of freedom for a cannulated pin guide that can be used to allow for selection and locking of an angle at which a target site can be reamed. The cannulation guide can include a coupling having a tab operatively coupled with the base where the coupling can be configured to receive the cannulated pin guide. The cannulated pin guide can pass through the coupling and into the base. The base can be configured to allow for movement of the coupling in a first direction while at the same time allowing for movement of the cannulated pin guide in a second direction different from the first direction thereby providing two degrees of freedom. When the cannulated pin guide is oriented at an angle preferred by a practitioner, an aperture 100 can be formed at a target site 102 using a cannulated pin guide, as shown in FIG. 1.

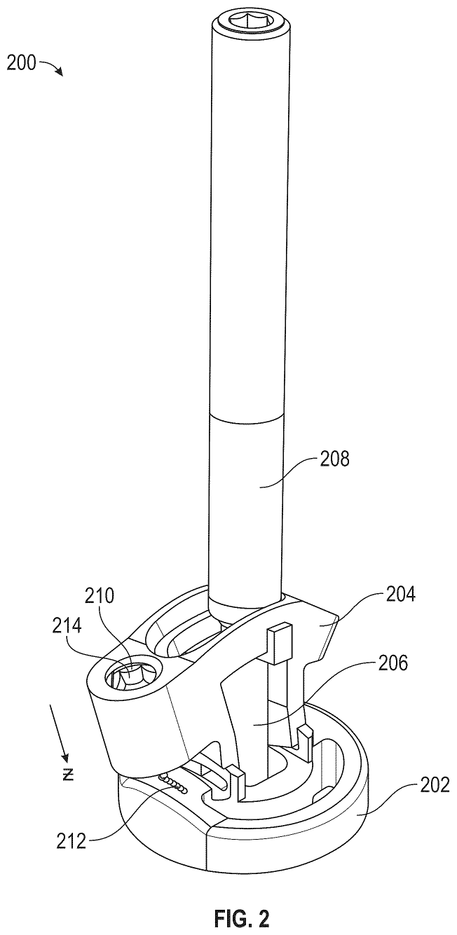

FIG. 1 illustrates an environment 104 where a cannulation guide 200 (FIG. 2) can be used to assist with the formation of the aperture 100 at the target site 102. The target site 102 can correspond to a scapula of a patient where a glenosphere 106 is placed at the target site 102 during a reverse shoulder replacement procedure. Preparation for the glenosphere 106 can include a pin 108 which can be inserted into the aperture 100. The glenosphere 106 can be configured to articulate with a complementary bearing surface attached to a stem 110 that can be implanted in a humerus 112 of the patient. In addition to positioning the aperture 100, the cannulation guide 200 can be used to facilitate the formation of the aperture 100. After a practitioner decides on the position and orientation of the aperture 100, the practitioner can use the cannulation guide 200 to form the aperture 100 during a drilling procedure.

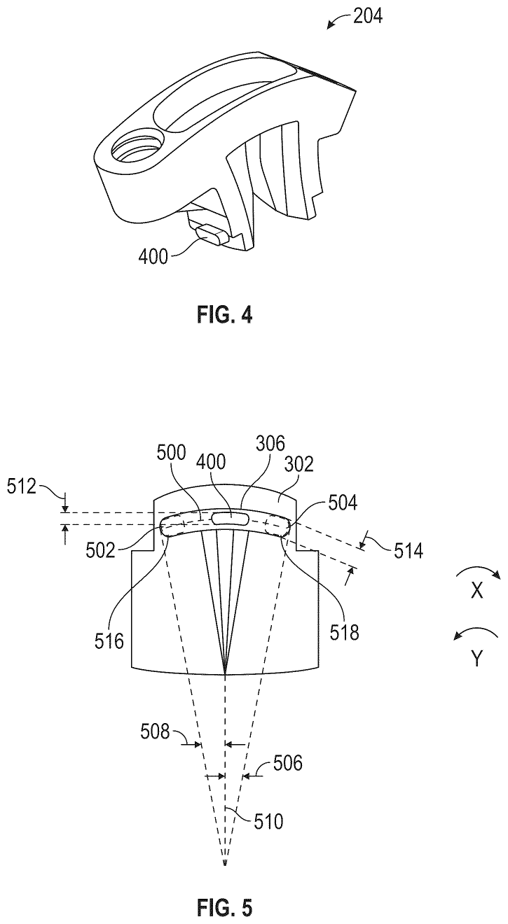

Now making reference to FIGS. 2-5, a schematic of the cannulation guide 200 is shown. The cannulation guide 200, which can be a medical device, can include a base 202 and a coupling 204 operatively coupled with the base 202. The cannulation guide 200 can also include a cannulated pin guide 206 that extends from the coupling 204 and into the base 202. Moreover, the cannulation guide 200 can have a cannulated pin guide handle 208 operatively coupled with the cannulated pin guide 206.

The base 202 can have a base cannula 300 through which the cannulated pin guide 206 can extend. In addition, the base 202 can have flanges 302 extending from a surface 304 of the base 202. The flanges 302 can have a base slot 306 through which a tab 400 of the coupling 204 can extend. The base slot 306 can have an arcuate configuration as shown in FIG. 5. The base slot 306 can extend along a coupling tab path 500 having end points 502 and 504. The coupling tab path 500 can be defined by angles 506 and 508 in relation to a baseline 510. The angles 506 and 508 can be in a range from 1° to 15° relative to the baseline 510 and preferably from 1° to 10° relative to the baseline 510. The coupling tab path 500 can provide a first degree of freedom for the cannulation guide 200.

The coupling tab 400 can have a width 512 while the coupling tab path can have a width 514, which can be greater than the coupling tab width 512. The coupling tab width 512 can be wide enough to slide within the base slot 306. Thus, the coupling tab 400 can move along the coupling tab path 500 between the coupling tab path end points 502 and 504 in directions X and Y between a first cannulated pin guide position 516 and a second cannulated pin guide position 518. While two cannulated pin guide positions are described for ease of reference, examples envision any number of cannulated pin guide positions. By virtue of the base slot 306 having an arcuate configuration, the directions X and Y can define an arcuate motion as the coupling tab 400 moves along the coupling tab path 500.

A practitioner can move the coupling 204 along the directions X and Y with the cannulated pin guide handle 208, which can be considered a first direction for the cannulation guide 200 and can allow the practitioner to dial in an angle at which a glenosphere can extend from the target site 102. Furthermore, when the coupling 204 moves along the directions X and Y, the cannulated pin guide 206 can also move along the directions X and Y.

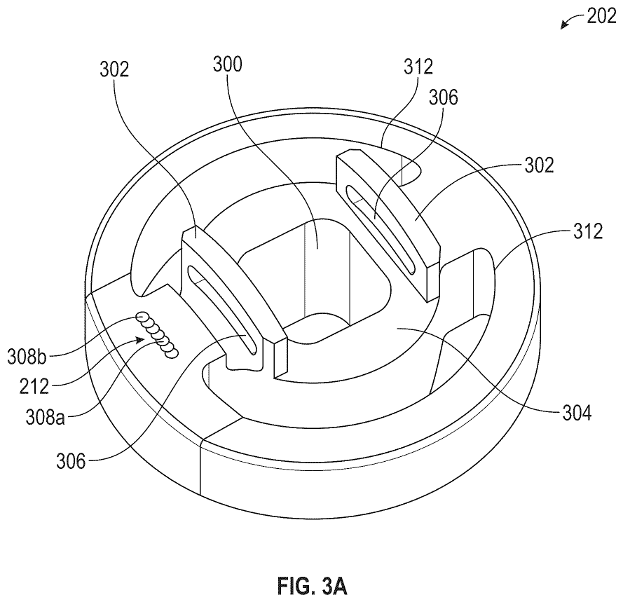

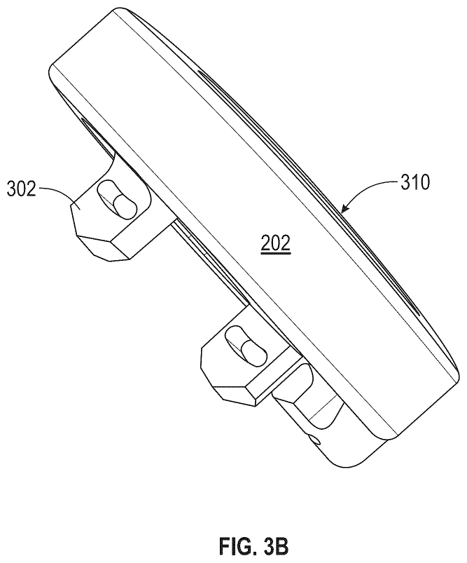

The base 202 can also have a bottom surface 310, which can have a curved configuration, as shown in FIG. 3B. The base bottom surface 310 can have a curved configuration to facilitate abutment against an area at which an aperture will be formed, such as the target site 102. While the base bottom surface 310 is shown as having a curved configuration, the base bottom surface 310 can have any configuration and can be dictated by a configuration of the target site 102. In particular, the base bottom surface 310 can have a concave configuration, a flat configuration, or the like.

The base 202 can be placed against the target site 102 during positioning of the cannulation guide 202 and formation of the aperture 100. The base 202 can include a window 312, which can allow a practitioner to view how the base 202 is resting on the target site 102.

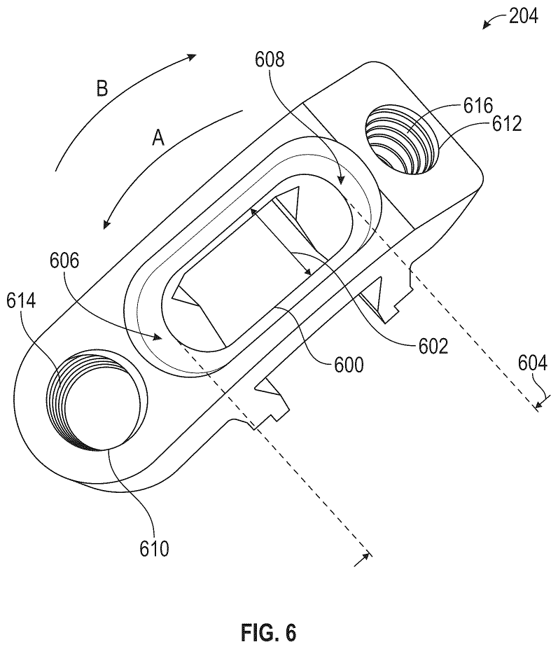

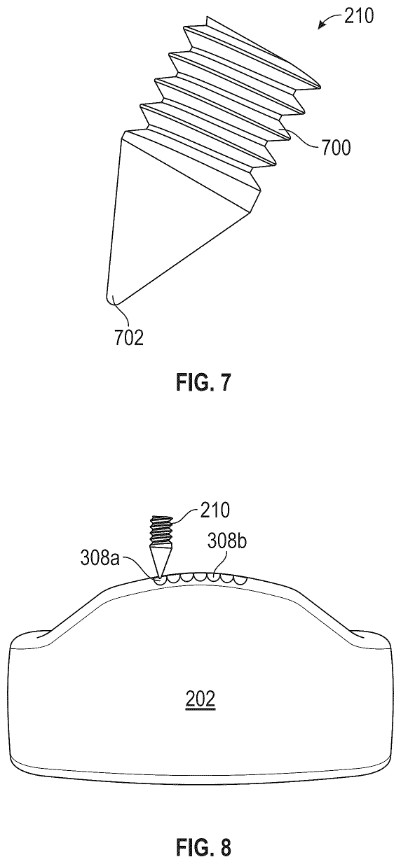

As mentioned above, the cannulated guide 200 can include the coupling 204. Now making reference to FIG. 6, the coupling 204 can include a slot 600 through which the cannulated pin guide 206 can pass. The coupling slot 600 can have a width 602 and be wide enough to have a sliding fit within the cannulation pin guide 206. in order to allow passage of the cannulated pin guide 206 to the base 202. Furthermore, when the cannulated pin guide 206 passes through the coupling slot 600, the cannulated pin guide 206 can move along directions A and B within the coupling slot 600. In order to allow movement along directions A and B, the coupling slot 600 can have length 604 that con be configured to allow the cannulated pin guide to achieve the angles 506 and 508.

More specifically, a practitioner can move the cannulated pin guide 206 between endpoints 606 and 608 of the coupling slot 600 along the directions A and B, which can be a second direction. In examples, the direction A can be perpendicular to the direction X and the direction B can be perpendicular to the direction Y. The first direction defined by the directions X and Y and second direction defined by the directions A and B can provide two degrees of freedom for the cannulation guide 200.

The coupling 204 can also include a set screw bore 610 and a handle bore 612. The set screw bore 610 can be configured to accept a set screw 210. In particular, the set screw bore 610 can include engagement means 614, such as threads, that are complementary to engagement means 700, such as threads, of the set screw 210. The set screw 210 can be disposed in the set screw bore 610 via the engagement means 614 and 700. Furthermore, the set screw can be adjusted in an up and down direction within the set screw bore 610 via the engagement means 614 and 700.

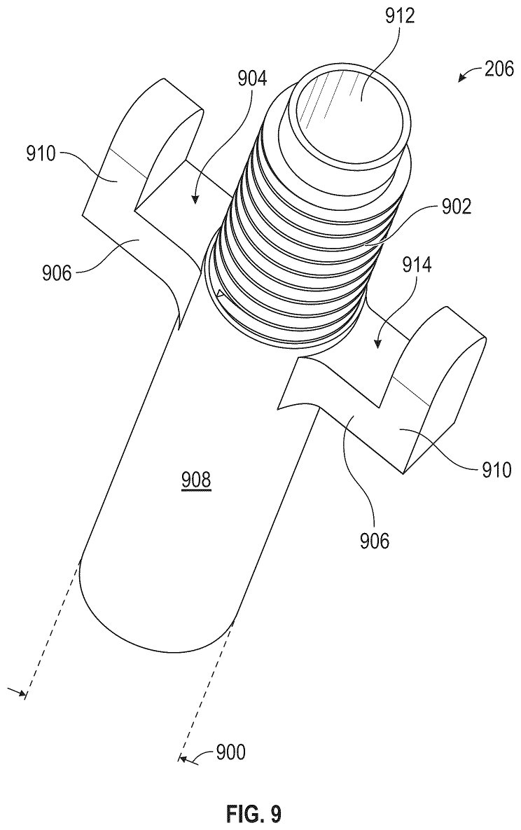

The set screw 210 can function to hold a position of the cannulated pin guide 206. In particular, the set screw 210 can include a tip 702, which can engage with first and second screw slots 308a and 308b of a screw set slot 212 of the base 202. Each of the first screw slot 308a and the second screw slot 308b can align with the set screw bore 610 in different positions based on the adjustment of the 204. As a practitioner moves the 204 along either the direction X or the direction Y, when the practitioner achieves a position of the 204 that is satisfactory, the practitioner can adjust the set screw 210 in a downward direction Z until the set screw tip 702 rests within the one of the slots 308a and 308b (FIG. 8). The practitioner can adjust the set screw 210 along the direction Z using a tool (not shown) that can engage with a socket 214 (FIG. 2). The practitioner can rotate the set screw 210 in either a clockwise or counter-clockwise direction, depending the configuration of the engagement means 616 and 700, in order to move the set screw 210 in the direction Z. The set screw 210 can continue to move in the direction Z until the set screw tip 702 rests within one of the slots 308a and 308b.

The handle bore 612 can include engagement means 616 that can engage with a handle (not shown). A practitioner can use the handle to move the coupling 204 along the directions X and Y when positioning the cannulated pin guide 206 at the target site 102.

With reference to FIG. 9, the cannulated pin guide 206 can have a width 900 that is less than the slot width 602 such that the cannulated pin guide 206 can move along the directions A and B while at the same time pass through the coupling slot 600 as described above. The cannulated pin guide 206 can also include engagement means 902, such as threads, that are configured to engage with engagement means disposed within the cannulated pin guide handle 208. Thus, the cannulated pin guide handle 208 can be secured to the cannulated pin guide 206 via the engagement means 902.

The cannulated pin guide 206 can include an opening 904 defined by a first wall 906 that extends from a stem 908 of the cannulated pin guide 206 and a second wall 910 that extends from the first wall 906, as shown in FIG. 9. As shown in FIG. 2, the coupling 204 can rest within the opening 904.

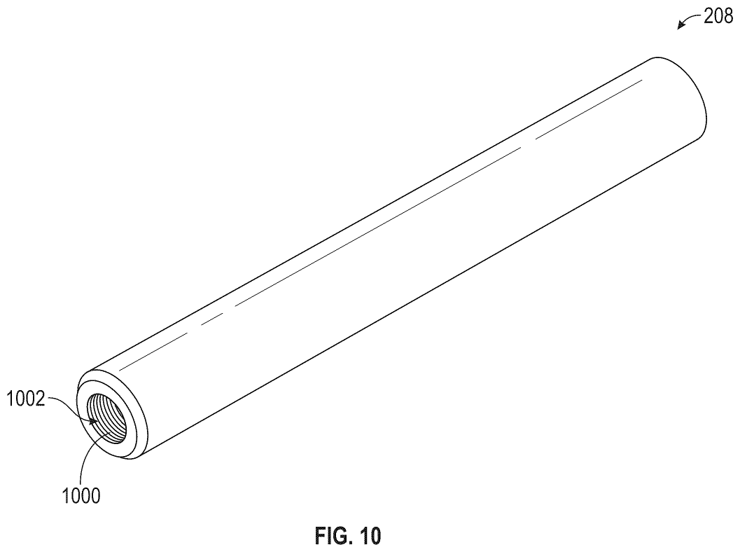

With reference to FIG. 10, the cannulated pin guide handle 208 can include engagement means 1000, such as threads, that are configured to engage with the engagement means 902 such that the cannulated pin guide handle 208 is secured with the cannulated pin guide 206. In particular, when the engagement means 902 couples with the engagement means 1000, the surfaces 904 and 914 can be compressed against the coupling 204, thereby locking the coupling 204. As mentioned above, a practitioner can use the cannulation guide 200 to form the aperture 100. In order to facilitate the formation of the aperture 100, the cannulated pin guide 206 can include a passageway 912 that can extend through the cannulated pin guide 206. The passageway 912 can align with a passageway 1002 of the cannulated pin guide handle 208. After a practitioner has decided on a location of the aperture 100, i.e., the practitioner has moved the cannulation guide 200 along the directions A, B, X, and/or Y, and secured the position of the cannulated pin guide 206 via the set screw 210 and on the slots 308a and 308b, the practitioner can insert a pin into the target site 102. Alternatively, the practitioner can feed an instrument, including a removal device such as a bone reamer, through the passageways 912 and 1002 and to the target site 102 in order to form the aperture 100.

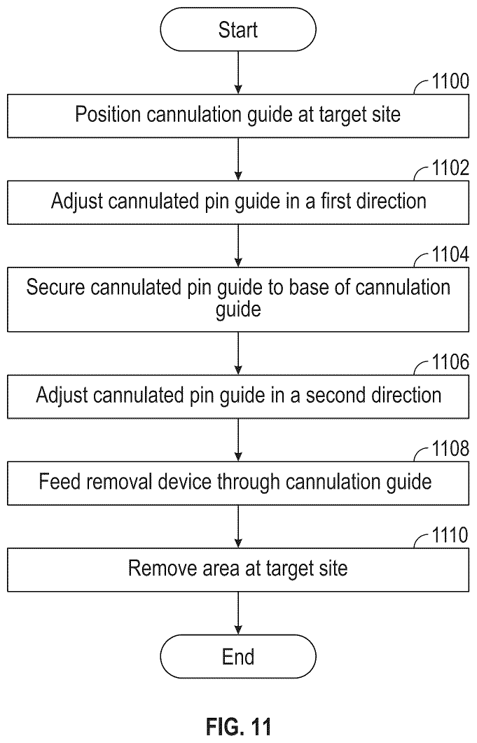

An example of using the cannulation guide 200 to form the aperture 100 is shown with reference to FIG. 11. Initially, during an operation 1100, a cannulation guide can be positioned at a target site. For example, the cannulation guide 200 can be positioned at the target site 102 where the base 202 can be positioned adjacent to the target site 102. More specifically, the target site 102 can have a curved configuration while the base bottom surface 310 can have the curved configuration that is complementary to the curved configuration of the target site 102. Thus, the base bottom surface 310 can be placed against the target site 102 when the cannulation guide 200 is positioned at the target site 102 during the operation 1100.

After the cannulation guide is positioned at the target site during the operation 1100, an operation 1102 can be performed where a cannulated pin guide is adjusted in a first direction. Once the cannulated pin guide is adjusted in the first direction, the cannulated pin guide can be secured to a base of the cannulation guide during an operation 1104. Returning to the example, during the operation 1102, the 204 can be adjusted in the direction Y into the position shown in FIG. 8 during the operation 1102. Afterwards, the set screw can be rotated counterclockwise along the direction Z such that the set screw tip 702 engages with the first set screw slot 308a, thereby securing the 204 to the base 202 of the cannulation guide 200.

After the cannulated pin guide is secured during the operation 1104, the cannulated pin guide can be adjusted in a second direction that is different from the first direction during an operation 1106. While the operation 1106 is described as being performed after the cannulated pin guide is secured in the operation 1104, the operation 1106 can be performed before the cannulated pin guide is secured to the base. Thus, the cannulated pin guide can be adjusted in both the first and second directions and secured after being adjusted in both directions. Moreover, while the cannulated pin guide is described as being adjusted in two directions, in some examples, the cannulated pin guide can be adjusted in just one direction instead of two directions.

In the example, during the operation 1106, the cannulated pin guide 206 can be adjusted in the direction A, where cannulated pin guide 206 is moved through the coupling slot 600. Once the cannulated pin guide is adjusted and positioned such that an aperture can be formed at the target site to accept a glenosphere pin, operations 1108 and 1110 can be performed. In the operation 1108, a removal device, such as bone reamer, can be fed through the cannulation guide to the target site 102 and an area at the target site 102 can be removed during the operation 1110.

In the example, a bone reamer (not shown) can be fed through the cannulated pin guide handle passageway 1002 and into the cannulated pin guide passageway 912. The bone reamer can then be fed through the cannulated pin guide passageway 912 and to the target site 102. Once at the target site 102, bone can be removed to form the aperture 100.

Having described various aspects and features of the inventive subject matter, the following numbered examples are provided as illustrative embodiments:

Example 1 is a medical device comprising: a base that includes: a cannula; and a flange extending from a surface of the base, the flange including a slot; a coupling operatively coupled with the base, the coupling having a tab extending therefrom and into the base flange, the coupling defining a slot; and a cannulated pin guide extending through the coupling slot and into the base cannula.

In Example 2, the subject matter of Example 1 includes, wherein the base slot has a width greater than a width of the coupling tab such that the coupling tab moves within the base slot in a first direction between a first coupling position and a second coupling position.

In Example 3, the subject matter of Example 2 includes, wherein the coupling slot has a width greater than a circumference of the cannulated pin guide such that the cannulated pin guide moves within the coupling slot in a second direction different from the first direction between a first cannulated pin guide position and a second cannulated pin guide position.

In Example 4, the subject matter of Example 3 includes, wherein the first direction is perpendicular to the second direction.

In Example 5, the subject matter of Examples 2-4 includes, wherein the base slot has an arcuate configuration such that the first direction defines an arcuate motion as the coupling tab moves within the base slot between the first coupling position and the second coupling position.

In Example 6, the subject matter of Examples 1-5 includes, the device further comprising a set screw.

In Example 7, the subject matter of Example 1-6 includes, wherein the coupling further includes set screw bore configured to accept the set screw.

In Example 8, the subject matter of Example 1-7 includes, wherein the base further includes a first screw slot and second screw slot that each align with the set screw bore in different positions, where a set screw tip of the set screw extends from the set screw bore and into one of the first screw slot and the second screw slot.

In Example 9, the subject matter of Example 1-8 includes, wherein the set screw tip extends from the set screw bore and into the first screw slot when the cannulated pin guide is in a first cannulated pin guide position and wherein the set screw tip extends from the set screw bore and into the second screw slot when the cannulated pin guide is in a second cannulated pin guide position.

In Example 10, the subject matter of Examples 1-9 includes, a cannulated pin guide handle coupled with the cannulated pin guide.

Example 11 is an apparatus comprising means to implement of any of Examples 1-10.

Example 12 is a system to implement of any of Examples 1-10.

Example 13 is a cannulation guide comprising: a base that includes: a cannula; and a flange extending from a surface of the base, the flange including a slot; a coupling operatively coupled with the base, the coupling having a tab extending therefrom and into the base flange, the coupling defining a slot, wherein the base slot has a width greater than a width of the coupling tab such that the coupling tab moves within the base slot in a first direction between a first coupling position and a second coupling position; and a cannulated pin guide extending through the coupling slot and into the base cannula.

In Example 14, the subject matter of Example 13 includes, wherein the coupling slot has a length greater than a circumference of the cannulated pin guide such that the cannulated pin guide moves within the coupling slot in a second direction different from the first direction between a first cannulated pin guide position and a second cannulated pin guide position.

In Example 15, the subject matter of Example 14 includes, wherein the first direction is perpendicular to the second direction.

In Example 16, the subject matter of Examples 13-15 includes, wherein the base slot has an arcuate configuration such that the first direction defines an arcuate motion as the coupling tab moves within the base slot between the first coupling position and the second coupling position.

In Example 17, the subject matter of Examples 13-16 includes, the device further comprising a set screw, wherein the coupling further includes set screw bore through which the set screw extends.

In Example 18, the subject matter of Example 13-17 includes, wherein the base further includes a first screw slot and second screw slot that each align with the set screw bore in different positions, where a set screw tip of the set screw extends from the set screw bore and into one of the first screw slot and the second screw slot.

In Example 19, the subject matter of Example 13-18 includes, wherein the set screw tip of the set screw extends from the set screw bore and into the first screw slot when the cannulated pin guide is in a first cannulated pin guide position and wherein the set screw tip extends from the set screw bore and into the second screw slot when the cannulated pin guide is in a second cannulated pin guide position.

Example 20 is an apparatus comprising means to implement of any of Examples 13-19.

Example 21 is a system to implement of any of Examples 13-19.

Example 22 is a cannulation guide comprising: a base that includes: a cannula; and a flange extending from a surface of the base, the flange including a slot; a coupling operatively coupled with the base, the coupling having a tab extending therefrom and into the base flange, the coupling defining a slot, the base slot having a width greater than a width of the coupling tab such that the coupling tab moves within the base slot in a first direction between a first coupling position and a second coupling position, wherein the base slot has an arcuate configuration such that the first direction defines an arcuate motion as the coupling tab moves within the base slot between the first coupling position and the second coupling position; and a cannulated pin guide extending through the coupling slot and into the base cannula.

In Example 23, the subject matter of Example 22 includes, the device further comprising a set screw, wherein the coupling further includes set screw bore configured to accept the set screw and the base further includes a first screw slot and second screw slot that each align with the set screw bore in different positions, where a set screw tip of the set screw extends from the set screw bore and into one of the first screw slot and the second screw slot.

In Example 24, the subject matter of Example 22-23 includes, wherein the set screw tip of the set screw extends from the set screw bore and into the first screw slot when the cannulated pin guide is in a first cannulated pin guide position and wherein the set screw tip extends from the set screw bore and into the second screw slot when the cannulated pin guide is in a second cannulated pin guide position.

Example 25 is an apparatus comprising means to implement of any of Examples 22-24.

Example 26 is a system to implement of any of Examples 22-24.

The above detailed description includes references to the accompanying drawings, which form a part of the detailed description. The drawings show, by way of illustration, specific examples that may be practiced. These embodiments are also referred to herein as “examples.” Such examples may include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, 4.24, and 5). Similarly, numerical ranges recited herein by endpoints include subranges subsumed within that range (e.g., 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

The above description is intended to be illustrative, and not restrictive. For example, the above-described examples (or one or more aspects thereof) may be used in combination with each other. Other examples may be used, such as by one of ordinary skill in the art upon reviewing the above description. The Abstract is to allow the reader to quickly ascertain the nature of the technical disclosure and is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the examples should be determined with reference to the appended claims, along with the full scope of equivalents to which such claims are entitled.

What is claimed is:

1. A medical device comprising:
a base that includes:
a cannula; and
a flange extending from a surface of the base, the flange including a slot;
a coupling operatively coupled with the base, the coupling having a tab extending therefrom and into the base flange, the coupling defining a slot; and
a cannulated pin guide extending through the coupling slot and into the base cannula.

2. The medical device of claim 1, wherein the base slot has a width greater than a width of the coupling tab such that the coupling tab moves within the base slot in a first direction between a first coupling position and a second coupling position.

3. The medical device of claim 2, wherein the coupling slot has a width greater than a circumference of the cannulated pin guide such that the cannulated pin guide moves within the coupling slot in a second direction different from the first direction between a first cannulated pin guide position and a second cannulated pin guide position.

4. The medical device of claim 3, wherein the first direction is perpendicular to the second direction.

5. The medical device of claim 2, wherein the base slot has an arcuate configuration such that the first direction defines an arcuate motion as the coupling tab moves within the base slot between the first coupling position and the second coupling position.

6. The medical device of claim 1, the device further comprising a set screw.

7. The medical device of claim 6, wherein the coupling further includes set screw bore configured to accept the set screw.

8. The medical device of claim 7, wherein the base further includes a first screw slot and second screw slot that each align with the set screw bore in different positions, where a set screw tip of the set screw extends from the set screw bore and into one of the first screw slot and the second screw slot.

9. The medical device of claim 8, wherein the set screw tip extends from the set screw bore and into the first screw slot when the cannulated pin guide is in a first cannulated pin guide position and wherein the set screw tip extends from the set screw bore and into the second screw slot when the cannulated pin guide is in a second cannulated pin guide position.

10. The medical device of claim 1, further comprising a cannulated pin guide handle coupled with the cannulated pin guide.

11. A cannulation guide comprising:
a base that includes:
a cannula; and
a flange extending from a surface of the base, the flange including a slot;
a coupling operatively coupled with the base, the coupling having a tab extending therefrom and into the base flange, the coupling defining a slot, wherein the base slot has a width greater than a width of the coupling tab such that the coupling tab moves within the base slot in a first direction between a first coupling position and a second coupling position; and
a cannulated pin guide extending through the coupling slot and into the base cannula.

12. The cannulation guide of claim 11, wherein the coupling slot has a length greater than a circumference of the cannulated pin guide such that the cannulated pin guide moves within the coupling slot in a second direction different from the first direction between a first cannulated pin guide position and a second cannulated pin guide position.

13. The cannulation guide of claim 12, wherein the first direction is perpendicular to the second direction.

14. The cannulation guide of claim 11, wherein the base slot has an arcuate configuration such that the first direction defines an arcuate motion as the coupling tab moves within the base slot between the first coupling position and the second coupling position.

15. The cannulation guide of claim 11, the device further comprising a set screw, wherein the coupling further includes set screw bore through which the set screw extends.

16. The cannulation guide of claim 15, wherein the base further includes a first screw slot and second screw slot that each align with the set screw bore in different positions, where a set screw tip of the set screw extends from the set screw bore and into one of the first screw slot and the second screw slot.

17. The cannulation guide of claim 16, wherein the set screw tip of the set screw extends from the set screw bore and into the first screw slot when the cannulated pin guide is in a first cannulated pin guide position and wherein the set screw tip extends from the set screw bore and into the second screw slot when the cannulated pin guide is in a second cannulated pin guide position.

18. A cannulation guide comprising:
a base that includes:
a cannula; and
a flange extending from a surface of the base, the flange including a slot;
a coupling operatively coupled with the base, the coupling having a tab extending therefrom and into the base flange, the coupling defining a slot, the base slot having a width greater than a width of the coupling tab such that the coupling tab moves within the base slot in a first direction between a first coupling position and a second coupling position, wherein the base slot has an arcuate configuration such that the first direction defines an arcuate motion as the coupling tab moves within the base slot between the first coupling position and the second coupling position; and
a cannulated pin guide extending through the coupling slot and into the base cannula.

19. The cannulation guide of claim 18, the device further comprising a set screw, wherein the coupling further includes set screw bore configured to accept the set screw and the base further includes a first screw slot and second screw slot that each align with the set screw bore in different positions, where a set screw tip of the set screw extends from the set screw bore and into one of the first screw slot and the second screw slot.

20. The cannulation guide of claim 19, wherein the set screw tip of the set screw extends from the set screw bore and into the first screw slot when the cannulated pin guide is in a first cannulated pin guide position and wherein the set screw tip extends from the set screw bore and into the second screw slot when the cannulated pin guide is in a second cannulated pin guide position.
